(19) United States
(12) Patent Application Publication
(10) Pub. No.: US 2021/0142864 A1
(43) Pub. Date: May 13, 2021

(54) PROGNOSTIC INDICATORS OF POOR OUTCOMES IN PREGNANT METASTATIC BREAST CANCER COHORT

(71) Applicant: NANTOMICS, LLC, Culver City, CA (US)

(72) Inventors: Christopher Szeto, Culver City, CA (US); Stephen Charles Benz, Culver City, CA (US); Andrew Nguyen, Culver City, CA (US)

(21) Appl. No.: 16/622,860

(22) PCT Filed: Jun. 15, 2018
(86) PCT No.: PCT/US2018/037876

§ 371 Date: Dec. 13, 2019

Related U.S. Application Data
(60) Provisional application No. 62/521,267, filed on Jun. 16, 2017.
(60) Provisional application No. 62/594,345, filed on Dec. 4, 2017.

(51) Int. Cl. G16B 5/20; G16B 20/00; G16B 40/00; G16H 50/70; G16H 10/40; G16H 50/20; G16H 10/60; G06N 20/10

(52) U.S. Cl. CPC G16B 5/20; G16B 20/00; G16B 40/00; G06N 20/10; G16H 10/40; G16H 50/20; G16H 10/60; G16H 50/70

(57) ABSTRACT

Transcriptomics data from tumor tissue of patients diagnosed with metastatic breast cancer are clustered and associated with overall survival of the patients. A subset of genes from one of the cluster associated with poor outcome are used to generate a survival prediction model predicting a survival time based on expression levels of a plurality of genes. Using such generated survival prediction model, a survival time of a patient diagnosed with metastatic breast cancer can be predicted and a treatment regimen can be updated or generated based on the survival time.

This application claims priority to our co-pending U.S. provisional applications with the Ser. No. 62/521,267, filed Jun. 16, 2017, and Ser. No. 62/594,345, filed Dec. 4, 2017.

### FIELD OF THE INVENTION

The field of the invention is systems and methods of identifying molecular profile of metastatic breast cancer that can be used to predict prognosis and/or survival of metastatic breast cancer patients.

### BACKGROUND OF THE INVENTION

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Upon first diagnosis, breast cancer is typically classified using various criteria, including grade, stage, and histopathology. Over the recent decade, molecular characterization was also increasingly taken into account and typically include receptor status, and particularly estrogen receptor (ER), progesterone receptor (PR), and human epidermal growth factor receptor 2 (HER2). In addition, numerous gene-based tests have become common to further subtype the cancer.

For example, efforts have been undertaken to refine triple negative breast cancer (TNBC) into molecular subtypes into several molecularly distinct subgroups based on retrospective analysis of observed treatment responses to chemotherapy (see e.g., PLOS ONE | DOI:10.1371/journal.pone.0157368 Jun. 16, 2016). Similarly, subtypes for TNBC were defined based on five potential clinically actionable groupings of TNBC: 1) basal-like TNBC with DNA-repair deficiency or growth factor pathways; 2) mesenchymal-like TNBC with epithelial-to-mesenchymal transition and cancer stem cell features; 3) immune-associated TNBC; 4) luminal/apocrine TNBC with androgen-receptor overexpression; and 5) HER2-enriched TNBC (see e.g., Oncotarget, Vol. 6, No. 15; pp 12890-12908). In yet another study (see e.g., J Breast Cancer 2016 September; 19(3): 223-230), subtypes of TNBC were identified as basal-like, mesenchymal, luminal androgen receptor, and immune-enriched. In still further known studies, expression subtyping was performed and identified three sub-clusters among tested patient samples (see e.g., Breast Cancer Research (2015) 17:43). Likewise, an online classification tool was published to classify TNBC by gene expression (URL: cbc.mc.vanderbilt.edu/tnbc; Cancer Informatics 2012:11 147-156) that separated TNBC data into six distinct subtypes.

However, where the breast cancer is metastatic breast cancer, patients often have a very unfavorable prognosis, despite novel targeted therapies. Moreover, prognostic and predictive factors for patients with advanced/metastatic breast cancer are not well understood. Indeed, a molecular assessment of patients and tumors in a metastatic setting is not routinely performed, despite advances in molecular precision medicine indicating great benefit to this patient group.

Thus, even though various systems and methods for classification of breast cancer are known in the art, molecular characterization of metastatic breast cancer is not well understood. As such, there remains a need for systems and methods that allow for molecular characterization of metastatic breast cancer.

### SUMMARY OF THE INVENTION

The inventive subject matter is directed to various systems and methods for using gene expression profiles of metastatic breast cancer tissues to identify clusters of genes that are significantly associated with overall survival time of patients. Such identified clusters can then be used to generate a survival prediction model, which predicts a survival time based on expression levels of a plurality of genes in the at least one cluster that is associated with a poor survival of at least some of the plurality of patients.

Thus, one aspect of the inventive subject matter includes a method of generating a survival prediction model for metastatic breast cancer. This method comprises a step of obtaining transcriptomics data of a plurality of patients diagnosed with metastatic breast cancer. The transcriptomics data into a plurality of clusters is then clustered into a plurality of clusters using complete Pearson correlation. Typically, the transcriptomics data comprises RNA-seq data and/or RNA expression levels of at least 1,000 genes, and number of clusters is determined using elbow method. Among the plurality of clusters, at least one cluster is identified as being associated with a poor survival of at least some of the plurality of patients by correlating the plurality of clusters with overall survival of the plurality of patients. Preferably, the plurality of clusters is differentially correlated with the overall survival of the plurality of patients. Then, the survival prediction model predicting a survival time based on expression levels of a plurality of genes is generated. Preferably, the plurality of genes is in the at least one cluster that is associated with a poor survival of at least some of the plurality of patients, and comprises at least one gene associated with WNT signaling pathway or pluripotency pathway. Also, it is preferred the at least one cluster has a hazard ratio is higher than 1.3.

Preferably, the plurality of genes are selected among the at least one cluster's transcriptomics data based on a quality of separation of high survivors from low survivors among the plurality of patients in a function of the expression levels of the plurality of genes. In some embodiments, the plurality of genes is less than 50. In other embodiments, the plurality of genes are selected from a group consisting of TMEM257, FAM180B, WNT11, CTDSPL, PROK1, GAD2, GRK7, FZD6, KRTAP505, KRT31, PRAMEF12, SYNGR4, SOX2, BHLHA9, POU1F1, KHNYN, CACNA2D4, C3orf36, RHOXF2, PABPN1L, EID2B, BBS4, AGPS, EFCC1, ROBO2, CMTM4, THTPA, ZP4, HIST1H2BE, LOC286238, IFNL2, DGKK, GNGT1, USP17L30, and ERN 1.

Additionally, the method may further include calculating concordance-index of the survival prediction model by comparing the predicted survival time with an actual survival time of the patients. Preferably, concordance-index of the survival prediction model is higher than 0.7.

In another aspect of the inventive subject matter, the inventors contemplate a method of predicting a survival time of a patient diagnosed with metastatic breast cancer. In this method, transcriptomic data of a tumor tissue of the patient is obtained and RNA expression levels of a plurality of genes from the transcriptomics data are determined. Typically, the transcriptomics data comprises RNA-seq data. Using a survival prediction model, the survival time of the patient can be predicted based on the RNA expression levels. Most preferably, at least two genes among the plurality of genes are associated with Wnt signaling pathway or pluripotency pathway.

Most typically, number of the plurality of genes is less than 50. Preferably, the plurality of genes are selected from a group consisting of TMEM257, FAM180B, WNT11, CTDSPL, PROK1, GAD2, GRK7, FZD6, KRTAP505, KRT31, PRAMEF12, SYNGR4, SOX2, BHLHA9, POU1F1, KHNYN, CACNA2D4, C3orf36, RHOXF2, PABPN1L, EID2B, BBS4, AGPS, EFCC1, ROBO2, CMTM4, THTPA, ZP4, HIST1H2BE, LOC286238, IFNL2, DGKK, GNGT1, USP17L30, and ERN1.

Preferably, survival prediction model is generated by obtaining transcriptomics data of a plurality of patients diagnosed with metastatic breast cancer. Then, the transcriptomics data into a plurality of clusters is then clustered into a plurality of clusters using complete Pearson correlation. Typically, the transcriptomics data comprises RNA-seq data and/or RNA expression levels of at least 1,000 genes, and number of clusters is determined using elbow method. Among the plurality of clusters, at least one cluster is identified as being associated with a poor survival of at least some of the plurality of patients by correlating the plurality of clusters with overall survival of the plurality of patients. Preferably, the plurality of clusters is differentially correlated with the overall survival of the plurality of patients. The plurality of genes used to predict the survival time in this method can be selected from the at least one cluster based on a quality of separation of high survivors from low survivors among the plurality of patients in a function of the expression levels of the plurality of genes. Also, it is preferred the at least one cluster has a hazard ratio is higher than 1.3.

Additionally, a concordance-index of the survival prediction model can be calculated by comparing the predicted survival time with an actual survival time of the patients. Preferably, concordance-index of the survival prediction model is higher than 0.7.

Further, the method may include a step of updating or generating a patient record based on the predicted survival time and/or modifying a treatment regimen for the patient based on the predicted survival time.

In still another aspect of the inventive subject matter, the inventors contemplate a method of generating or updating a treatment regimen for a patient diagnosed with metastatic breast cancer. In this method, transcriptomic data of a tumor tissue of the patient is obtained and RNA expression levels of a plurality of genes from the transcriptomics data are determined. Typically, the transcriptomics data comprises RNA-seq data. Then, using a survival prediction model, the survival time of the patient can be predicted based on the RNA expression levels. The method continues with a step of generating or updating the treatment regimen to include at least one agent targeting a pathway element of Wnt signaling pathway or pluripotency pathway.

Most typically, number of the plurality of genes is less than 50. Preferably, the plurality of genes are selected from a group consisting of TMEM257, FAM180B, WNT11, CTDSPL, PROK1, GAD2, GRK7, FZD6, KRTAP505, KRT31, PRAMEF12, SYNGR4, SOX2, BHLHA9, POU1F1, KHNYN, CACNA2D4, C3orf36, RHOXF2, PABPN1L, EID2B, BBS4, AGPS, EFCC1, ROBO2, CMTM4, THTPA, ZP4, HIST1H2BE, LOC286238, IFNL2, DGKK, GNGT1, USP17L30, and ERN1. Alternatively, the plurality of genes includes WNT11, SOX2, and FZD6.

Preferably, survival prediction model is generated by obtaining transcriptomics data of a plurality of patients diagnosed with metastatic breast cancer. Then, the transcriptomics data into a plurality of clusters is then clustered into a plurality of clusters using complete Pearson correlation. Typically, the transcriptomics data comprises RNA-seq data and/or RNA expression levels of at least 1,000 genes, and number of clusters is determined using elbow method. Among the plurality of clusters, at least one cluster is identified as being associated with a poor survival of at least some of the plurality of patients by correlating the plurality of clusters with overall survival of the plurality of patients. Preferably, the plurality of clusters is differentially correlated with the overall survival of the plurality of patients. The plurality of genes used to predict the survival time in this method can be selected from the at least one cluster based on a quality of separation of high survivors from low survivors among the plurality of patients in a function of the expression levels of the plurality of genes. Also, it is preferred the at least one cluster has a hazard ratio is higher than 1.3.

Additionally, a concordance-index of the survival prediction model can be calculated by comparing the predicted survival time with an actual survival time of the patients. Preferably, concordance-index of the survival prediction model is higher than 0.7. Further, the method may include a step of updating or generating a patient record based on the predicted survival time.

Various objects, features, aspects and advantages of the inventive subject matter will become more apparent from the following detailed description of preferred embodiments, along with the accompanying drawings.

### BRIEF DESCRIPTION OF THE DRAWING

FIG. 1 is a schematic illustration of the PRAEGNANT study program.

FIG. 2 is a graph depicting overall survival (OS) in the PRAEGNANT study program as stratified by immunohistochemical (IHC) grouping.

FIG. 3 is a graph depicting overall survival (OS) in the PRAEGNANT study program as stratified by PAM50 subtype grouping.

FIG. 4 is an exemplary heat map for the 1,000 most variantly expressed genes and clustering into five clusters using complete Pearson correlation.

FIG. 5 is a graph depicting overall survival (OS) in the PRAEGNANT study program as stratified by gene expression levels of five clusters of genes determined in FIG. 4.

FIGS. 6A and 6B show exemplary Venn diagram graphs for poorest survival groupings (6A) and best survival groupings (6B) in clusters 5 and 2, respectively.

FIG. 7 shows an exemplary time-to-death prediction graph with training data set and evaluating data set.

FIG. 8 shows a heat map of the 35 genes used in the survival prediction model.

### DETAILED DESCRIPTION

The inventors has now discovered that expression profiling of genes determined from tumor tissue of patients diagnosed with metastatic breast cancer can be used to generate clusters of gene expression patterns that are associated with different levels of overall survival of metastatic breast cancer patients. The inventors further discovered that such generated clusters, more specifically a high-risk cluster that is associated with poor prognosis or poor survival of the metastatic breast cancer patients could be a better indicator than other markers or subtyping methods to predict a survival time or a time-to-death of patients with bad prognosis. Among the genes in the high-risk cluster, the inventors could identify a small subset of genes that are most substantially associated with survival time, which can be used to generate a prediction model with high accuracy.

Viewed from a different perspective, the inventors discovered that a survival time or a time-to-death of patients can be more reliably predicted by determining expression profiling of a group of genes that were identified by clustering the transcriptomics into a plurality of clusters that are associated different survival time or a time-to-death of patients. The inventors further found that the number of genes of the group of genes can be reduced using machine learning while maintaining or even increasing the reliance and accuracy of the prediction to so reduce the amount of data processed to provide accurate prediction of survival time of a patient. Consequently, in one especially preferred aspect of the inventive subject matter, the inventors contemplate a method of generating a survival prediction model for metastatic breast cancer using transcriptomics data of a plurality of patients diagnosed with metastatic breast cancer and clustering the transcriptomics data into a plurality of clusters, at least one of which is associated with a poor survival of patients. A subset of genes, and/or its expression pattern from such clustered transcriptomics data can be identified and associated with overall survival to so generate a reliable survival prediction model.

As used herein, the term “tumor” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

### Obtaining Transcriptomics Data

Any suitable methods and/or procedures to obtain omics data, especially transcriptomics data are contemplated. For example, the transcriptomics data can be obtained by obtaining tissues from an individual and processing the tissue to obtain RNA from the tissue to further analyze relevant information. In another example, the transcriptomics data can be obtained directly from a database that stores transcriptomics information of an individual.

Where the omics data is obtained from the tissue of an individual, any suitable methods of obtaining a tumor sample (tumor cells or tumor tissue) or healthy tissue from the patient are contemplated. Most typically, a tumor sample or healthy tissue sample can be obtained from the patient via a biopsy (including liquid biopsy, or obtained via tissue excision during a surgery or an independent biopsy procedure, etc.), which can be fresh or processed (e.g., frozen, etc.) until further process for obtaining omics data from the tissue. For example, tissues or cells may be fresh or frozen. In other example, the tissues or cells may be in a form of cell/tissue extracts. In some embodiments, the tissues or cells may be obtained from a single or multiple different tissues or anatomical regions. For example, a metastatic breast cancer tissue can be obtained from the patient's breast as well as other organs (e.g., liver, brain, lymph node, blood, lung, etc.) for metastasized breast cancer tissues. In another example, a healthy tissue or matched normal tissue (e.g., patient's non-cancerous breast tissue) of the patient can be obtained from any part of the body or organs, preferably from liver, blood, or any other tissues near the tumor (in a close anatomical distance, etc.).

In some embodiments, tumor samples can be obtained from the patient in multiple time points in order to determine any changes in the tumor samples over a relevant time period. For example, tumor samples (or suspected tumor samples) may be obtained before and after the samples are determined or diagnosed as cancerous. In another example, tumor samples (or suspected tumor samples) may be obtained before, during, and/or after (e.g., upon completion, etc.) a one time or a series of anti-tumor treatment (e.g., radiotherapy, chemotherapy, immunotherapy, etc.). In still another example, the tumor samples (or suspected tumor samples) may be obtained during the progress of the tumor upon identifying a new metastasized tissues or cells.

From the obtained tumor samples (cells or tissue) or healthy samples (cells or tissue), RNA (e.g., mRNA, miRNA, siRNA, shRNA, etc.) can be isolated and further analyzed to obtain transcriptomics data. Alternatively and/or additionally, a step of obtaining transcriptomics data may include receiving transcriptomics data from a database that stores transcriptomics information of one or more patients and/or healthy individuals. For example, transcriptomics data of the patient's tumor may be obtained from isolated RNA from the patient's tumor tissue, and the obtained omics data may be stored in a database (e.g., cloud database, a server, etc.) with other transcriptomics data set of other patients having the same type of tumor or different types of tumor. Transcriptomics data obtained from the healthy individual or the matched normal tissue (or healthy tissue) of the patient can be also stored in the database such that the relevant data set can be retrieved from the database upon analysis.

Transcriptomics data of cancer and/or normal cells comprises sequence information and/or expression level (including expression profiling, copy number, or splice variant analysis) of RNA(s) (preferably cellular mRNAs) that is obtained from the patient, from the cancer tissue (diseased tissue) and/or matched healthy tissue of the patient or a healthy individual. There are numerous methods of transcriptomic analysis known in the art, and all of the known methods are deemed suitable for use herein (e.g., RNAseq, RNA hybridization arrays, qPCR, etc.). Consequently, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis, especially including RNAseq, qPCR and/or rtPCR based methods, although various alternative methods (e.g., solid phase hybridization-based methods) are also deemed suitable.

It should be appreciated that one or more desired nucleic acids or genes may be selected for a particular disease (e.g., cancer, etc.), disease stage, or types of analysis. Preferably, the transcriptomics data comprises RNA expression levels of variably expressed genes. As used herein, the variably expressed gene refer any gene whose expression level varies among samples at least 10%, preferably at least 20%, more preferably at least 30%, most preferably at least 50%. Thus, the numbers of the genes that are included in the transcriptomics data may vary depending on the particular disease (e.g., cancer, etc.), disease stage, or types of analysis. Most typically, in transcriptomics data of metastatic breast cancer tissues, the number of variably expressed genes to be included in the transcriptomics data is at least 300 genes, preferably at least 5,00 genes, more preferably at least 1,000 genes, and most preferably at least 1,500 genes.

One exemplary protocol and/or database of obtaining transcriptomics data from patients may include a prospective molecular breast cancer registry (PRAEGNANT; study protocol (NCT02338767)) that includes completed transcriptomic profiling and is designed to provide an infrastructure for real-time comprehensive analysis of tumor/patient molecular characteristics. As shown in FIG. 1, the PRAEGNANT study program focuses on patients with either metastasis or inoperable loco-regional disease. Inclusion is not limited to patients receiving specific treatment lines. Disease progression must be objectively evaluable. Tumor reevaluation is done every 2-3 months, with additional assessments carried out if disease continues to progress and after every change of treatment. Adverse events and severe adverse events are continually reported throughout the study as is quality of life, and a program (PRO; Patient-reported Outcomes) is used which allows patients to document their quality of life themselves together with any adverse events.

### Transcriptomics Analysis and Clustering

The inventors contemplate that transcriptomics data of a plurality of patients diagnosed with the same disease, preferably in the similar stage of the disease, can be clustered into multiple groups based on the correlations and/or pattern of expression levels of genes. Any suitable methods of clustering the transcriptomics data are contemplated. For example, the variably expressed genes in tumor tissues can be clustered using a linear regression method, preferably using complete Pearson correlation. In such example, it is preferred that the absolute value of the correlation coefficient in one group or cluster of genes is more than at least 0.4, preferably more than 0.5, more preferably more than 0.6, most preferably more than 0.7. Thus, in some scenarios, the genes in one cluster or one group can be divided into two or more subgroups that are negatively or positively correlated with each other.

In addition, numbers (quantities) of clusters or groups (e.g., k in k-means algorithm) can be determined by any suitable means or algorithms. One exemplary and preferred method is elbow method. Yet, other methods including x-means clustering, information criterion approach (e.g., Akaike information criterion (AIC), Bayesian information criterion (BIC), or the Deviance information criterion (DIC), etc.), information-theoretic approach (e.g., jump method, etc.), the silhouette method, and/or cross-validation method. Where the elbow method is used to determine the number of clusters, it is preferred that the gain of the percentage of variance explained (F-test value) with the determined number value and the next value is less than 10%, or preferably less than 5%. For example, as shown in FIG. 4, in a heat map, over 1,000 variably expressed genes are clustered into five clusters based on the gene expression patterns using complete Pearson correlation. The optimal number of clusters between 3 and 10 was identified using the elbow method (data not shown), and k-means was used to associate transcriptomics data (gene expression levels) of each tumor sample of each patient (total 142 samples) with one of five clusters.

It is contemplated that each cluster of transcriptomics data can be associated with differential overall survival of patients, and at least one cluster that is associated with a poor survival can be identified. As used herein, overall survival is measured by number of days from the date of diagnosis that patients diagnosed with the disease are still alive. For example, as shown in FIG. 5, overall survival of subsets of patients corresponding to each cluster (clusters 1-5), as visualized on a Kaplan Meier curve, shows differential overall survival among five clusters. A Cox proportional hazard model was fit to these five clusters and hazard ratio of each cluster was calculated from the association coefficients. Generally, hazard ratios can be calculated based on the number of variably expressed genes (number of covariants) and the impact of variably expressed genes. The inventors found that among five clusters, cluster 5 (corresponding to transcriptomics data of total 13 samples) has highest hazard ratio (1.451, p=0.0021), indicating that cluster 5 is most significantly associated with poor outcome of the metastatic breast cancer prognosis.

The inventors found that overall survival of patients, especially the poor outcome of the patients, is more significantly associated with clustered genes and their expression patterns compared to other individual clinical features or markers known to be associated with the metastatic breast cancer. For example, tumor tissues were obtained from a plurality of metastatic breast cancer patients according to the experimental scheme as shown in FIG. 1. Based on early results available, twenty-five clinical features were tested independently in Cox-proportional hazard models for significant association with survival as is exemplarily shown in Table 1. Features included diagnosis information (grade, hormone receptor status, etc.), health correlates (BMI, weight, etc.), personal and family history of prior breast cancer diagnoses, among others. Among such features, the inventors identified five features (estrogen receptor (ER) or progesterone receptor (PR) positive, Triple-negative status, Diagnostic before 61 and triple-negative status, PR positive status, and body mass index (BMI)) that were significantly associated with differential survival (p<0.05), as well as three additional features (ER status, HER2 status, and grade at diagnosis) used to define subtypes. The strongest indicators of outcome were molecular characteristics: ER or PR positive status and triple-negative status (ER−PR−HER2−).

TABLE 1

| | Hazard | |
|---|---|---|
| | Ratio | p-value |
| ER or PR positive | 0.704 | 0.0052 |
| Triple-negative status (TNBC) | 1.360 | 0.0093 |
| Diagnosis before 61 and TNBC | 1.306 | 0.0215 |
| PR status | 0.728 | 0.0255 |
| Body mass index (BMI) | 0.682 | 0.0340 |
| ER status | 0.802 | 0.1161 |
| HER2 status | 0.821 | 0.2797 |
| Grade at diagnosis | 1.137 | 0.4578 |

Thus, next, the inventors evaluated the correlations between the molecular markers and clinical subtypes of the metastatic breast cancer and overall survival rate using three immunohistochemical (IHC) markers for metastatic breast cancer: estrogen receptor (ER), progesterone receptor (PR) and epidermal growth factor (HER2), along with grade at diagnosis (G1) to define clinical subtypes. Patient's biopsy tissues were obtained and the expression and/or intensity of marker proteins were determined to group the patient's samples into four groups or clusters: IHC negative for all three receptors are grouped as TNBC; HER2+ samples are grouped as HER2; ER/PR+ and G1 less than 3 were grouped as Luminal A; ER/PR+ and G1 more than 2 were grouped as Luminal B. Overall survival (OS) was plotted against the standard IHC classifications (Luminal A, Luminal B, TNBC, and HER2) as shown in FIG. 2. A Cox proportional hazard model was fit to these 4 groups and hazard ratios were calculated from the association coefficients. While the expected trends are apparent (e.g., TNBC has worse prognosis), the inventors could find that classification based on clinical and molecular subtypes (protein expression level) could not be associated with overall survival of the patients in a statistically significant level at the cohort size.

The inventors further determined whether correlations between the clinical and molecular subtypes with the overall survival of the patient are more substantial when the clinical and molecular subtypes are analyzed with their transcriptomics data. Thus, known clinical correlates for OS (e.g. hormone-receptor status, age at diagnosis, and BMI) were analyzed by Cox proportional hazard ratios, and compared to transcriptomic markers of outcomes. All patient tumors were sequenced on the Illumina sequencing platform, and RNAseq expression data was analyzed by RSEM to estimate transcripts per million (TPM) values for each gene isoform. Log-TPM values were used in established PAM50 intrinsic breast cancer cluster gene sets to identify subgroups in the PREAGNANT cohort. Overall survival (OS) was plotted against the standard PAM50 intrinsic subtypes: Luminal A, Luminal B, Basal, and HER2 as shown in FIG. 3. A Cox proportional hazard model was fit to these 4 subgroups and hazard ratios were calculated the association coefficients. The inventors found that while the HER2 group did not have sufficient representation for analysis, Basal and Luminal A subtypes were significantly associated with poor and best survival respectively. Based on the available omics data from the study protocol, the inventor found that hormone receptor positivity (HR=0.7, p<0.006) and TNBC status (HR=1.4, p<0.01) were significantly associated with outcomes. Moreover, PAM50 subtypes were also strong indicators of outcomes (e.g., Basal disease compared to other subtypes has HR=1.34, p<0.04). Notably, the expression-based PAM50 subtypes showed more significant differential survival than the equivalent IHC-based subtypes.

Yet, even though some PAM50 subtypes could be relatively strongly associated with overall survival of patients, the inventors found that RNA expression-based high-risk cluster in this cohort was more indicative of poor prognosis than clinical variants, IHC markers, or established subtypes, with a HR=1.45 (p<0.003) when compared to other clusters. Table 2 lists the patient subgroups having best and poorest overall survival using IHC/clinical information, established expression subtypes, and clustering using RNA expression levels of multiple genes among patient. The intrinsic subtypes (clustering using RNA expression levels of multiple genes) in this cohort are the most strongly associated with differential survival (p<0.02) compared to IHC/clinical subtypes or PAM50 intrinsic subtypes.

TABLE 2

| | Poorest | Best | Differential |
|---|---|---|---|
| | survival | survival | survival |
| | group | group | p-value (long-rank) |
| IHC/clinical subtypes | TNBC | LumA | 0.0923 |
| PAM50intrinsic subtypes | Basal | LumA | 0.0204 |
| PRAEGNANT | Cluster 5 | Cluster 2 | 0.0159 |
| intrinsic subtypes | | | |

Further, the inventors also found that the patients groups that are classified by IHC/clinical information, established expression subtypes (PAM50), and clustering using RNA expression levels of multiple genes among patient do not substantially overlap. For example, FIG. 6A shows a Venn diagram of three patients groups that are mostly associated with poor outcome of the metastatic breast cancer (TNBC group from IHC/clinical subgrouping, Basal group from PAM50 subgrouping, cluster 5 from clustering using RNA expression levels). While there is some overlapped patient population between or among three groups of poorest overall survival, none of two group combinations share more than 50% of patients of each group. Similarly, FIG. 6B shows a Venn diagram of three patients groups that are mostly associated with the best outcome of the metastatic breast cancer (LumA groups for IHC/clinical and PAM50, and cluster 2 from clustering using RNA expression levels). While there is some overlapped patient population between or among three groups of poorest overall survival, none of two group combinations share more than 50% of patients of each group. Further, even the group of patients classified as LumA group in IHC/clinical subgrouping and group of patients classified as LumA group in PAM50 subgrouping are not substantially overlapping, indicating that the subgrouping using same molecular markers (in different forms, either protein or RNA) in IHC/clinical subgrouping and PAM50 subgrouping may render different correlations of markers with overall survival, and thus unreliable prediction of survival time may be resulted using the correlations from such subgrouping.

Such results suggest that the molecular profiling by clustering the genes whose expression levels are correlated can be used to generate more accurate prediction model of overall survival of a patient or expected prognosis, especially of poor outcome of a patient diagnosed with metastatic breast cancer. Thus, the inventors further contemplate that at least one cluster generated from correlating RNA expression levels of genes can be selected to generate a survival prediction model using machine learning that predicts the survival time (or a time to death) in a function of the patient's RNA expression levels of a plurality of genes in the selected cluster. In a preferred embodiment, the gene cluster used to generate the survival prediction model is the one that is most substantially related to the poor outcome of patients. In another preferred embodiment, the gene cluster used to generate the survival prediction model has a hazard ratio higher than 0.8, preferably higher than 1.0, more preferably higher than 1.2, most preferably higher than 1.3. For example, the preferred cluster of genes of metastatic breast cancer may include cluster 5 shown in FIGS. 4 and 5 as that cluster is most substantially anti-correlated with the overall survival of metastatic breast cancer patients.

In some embodiments, the entire or substantially all genes in the selected cluster can be used to generate a survival prediction model. In such embodiments, it is preferred that the number of genes in the selected cluster is less than 200, preferably less than 100, more preferably less than 50 genes to efficiently process the data and also to reduce unreliably variable expression data. In other embodiments, a subset of genes among all genes in the cluster can be selected to generate a survival prediction model. In such embodiments, it is preferred that the subset of genes is selected based on a quality of separation of high survivors from low survivors among the plurality of patients in a function of the expression levels of the plurality of genes. In other words, for example, the subset of genes is selected when the metastatic breast cancer patients who survived long (top 10%, top 20%, top 30% with respect to the overall survival) have at least 10%, at least 20%, at least 30% higher or lower average expression level of the plurality of genes, overall or individually.

Alternatively and/or additionally, the subset of genes can be selected by machine learning algorithm that reduces the number of genes to maximize the predictability and efficiency of the survival prediction model. Generally, selection or reduction process allows determination of level of importance in each variable (e.g., each gene expression level, etc.) and also allows assessing the effects of other variables when such are eliminated statistically. Any suitable machine learning algorithms are contemplated, and exemplary machine learning algorithms include, but not limited to, Linear kernel support vector machine (SVM) (SVM as described in the publication entitled “A User's Guide to Support Vector Machines” by Ben-Hur et al., which is incorporated by reference herein in its entirety), First order polynomial kernel SVM, Second order polynomial kernel SVM, Ridge regression, Lasso, Elastic net, Sequential minimal optimization, Random forest, J48 trees, Naive bayes, JRip rules, HyperPipes, and NMFpredictor. In such example, it is contemplated that the prediction model can be generated and trained with at least 40%, at least 50%, at least 60%, at least 70% of the patients' transcriptomics data and survival data as training data set. The number of genes used to analyze the training data set and be selected for building the prediction model can be reduced using selection process (e.g., variance threshold selection, L1 selection, etc.). Then, the prediction model can be tested with a subset of the patients' transcriptomics data and survival data as evaluation data sets.

In some embodiments, the validity of the prediction model can be determined by calculating concordance index of the prediction model. Generally, concordance index or concordance frequency increases when the number of patient with matched predicted survival time and the actual survival time increases. Preferably, the survival time prediction model using the selected subset of genes and their expression levels has concordance index higher than 0.5, preferably higher than 0.6, more preferably higher than 0.7, most preferably higher than 0.75.

FIG. 7 shows one exemplary graph of plotting the training set's predicted overall survival data generated by the prediction model (shown as squares) and the evaluation data set's predicted overall survival data generated by the prediction model (round) and the actual survival data. Whole RNAseq Expression and survival data for forty-three patients that have an annotated death were used to build and test a time-to-death prediction model. Eighty-percent of these patients were randomly selected as the training set. The resulting model was applied to predicting OS in the held-out 20% test samples. This model achieved a 0.78 concordance index with true OS labels.

In the prediction model shown as graph in FIG. 7, the inventors found that the number of genes to generate the prediction model can be reduced to less than 50. More specifically, a Lasso regression model was fit to the training data, which uses an L1-selection process to minimize the number of genetic features utilized in the final predictive model resulting in a model that uses just 35 features down from >19K features (genes, gene expression levels, etc.). FIG. 8 shows a heat map the 35 genes used in this survival prediction model. Rows are sorted by hierarchical clustering, columns are sorted left to right in order of increasing OS. There is a clear pattern of differential expression between low and high survivors, including gene expression levels of TMEM257, FAM180B, WNT11, CTDSPL, PROK1, GAD2, GRK7, FZD6, KRTAP505, KRT31, PRAMEF12, SYNGR4, SOX2, BHLHA9, POU1F1, KHNYN, CACNA2D4, C3orf36, RHOXF2, PABPN1L, EID2B, BBS4, AGPS, EFCC1, ROBO2, CMTM4, THTPA, ZP4, HIST1H2BE, LOC286238, IFNL2, DGKK, GNGT1, USP17L30, and ERN1.

The inventors further found that some genes in the 35 selected genes used in the survival prediction model are associated with one or more tumor-associated pathways. 35 selected genes are analyzed using Gene-set enrichment analysis (GSEA). Table 3 depicts results for an exemplary GSEA for these 35 predictive genes. Five databases were queried against (Wikipathways, GO, KEGG, etc.) for curated gene sets enriched for these predictive genes. This table shows those significantly associated (adjusted p<0.05). Three of the 35 genes are consistently identified as associated with WNT signaling and pluripotency, suggesting a functional annotation for this prognostic model.

TABLE 3

| | | Adjusted | | |
|---|---|---|---|---|
| Term | Overlap | P-value | Genes | Database |
| Wnt Signaling Pathway and | 3/94 | 0.01647 | SOX2; WNT11:FZD6 | WikiPathways_2016 |
| Pluripotency_Mus musculus_WP723 | | | | |
| Wnt Signaling Pathway and | 3/102 | 0.01647 | SOX2; WNT11:FZD6 | WikiPathways_2016 |
| Pluripotency_Homo sapiens_WP399 | | | | |
| Phototransduction_Homo | 2/27 | 0.04322 | GNGT1; GRK7 | KEGG_2016 |
| sapiens_hsa04744 | | | | |
| Signaling pathways regulating | 3/142 | 0.04322 | SOX2; WNT11:FZD6 | KEGG_2016 |
| pluripotency of stem cells_Homo | | | | |
| sapiens_hsa04550 | | | | |
| Hippo signaling pathway_Homo | 3/153 | 0.04322 | SOX2; WNT11:FZD6 | KEGG_2016 |
| sapiens_hsa04390 | | | | |

It should be appreciated that the use of molecular profiling to develop prognostic signatures out-performs standard clinical correlates of poor outcomes in the metastatic setting, even in a small subset of the total cohort. In addition, the prediction model generated using such clustered gene expressions as group of markers, instead of a single or a few known clinical markers, could provide more reliable, highly accurate, predicted or estimated survival time to a patient diagnosed with metastatic breast cancer. Thus, this approach advances and improves the diagnostic and/or prognostic tool for metastatic breast cancer, whose prognosis could not be reliably predicted using the previous technology using a single or a few known clinical markers or phenotypes. Further, by identifying several tumor pathway-related genes among the subset of gens, this approach also provides potential targets to treat the metastatic breast cancer patients having poor outcomes.

Thus, in another aspect of the inventive subject matter, the inventors contemplate a method of predicting a survival time of a patient diagnosed with metastatic breast cancer. In this method, transcriptomics data of tumor tissue(s), either from a single anatomical location or a plurality of anatomical locations, are obtained. Among the transcriptomics data, a subset of transcriptomics data that is relevant to predict the survival time of the patient can be further obtained. Preferably, the subset of transcriptomics data includes RNA expression levels of a plurality of genes selected from TMEM257, FAM180B, WNT11, CTDSPL, PROK1, GAD2, GRK7, FZD6, KRTAP505, KRT31, PRAMEF12, SYNGR4, SOX2, BHLHA9, POU1F1, KHNYN, CACNA2D4, C3orf36, RHOXF2, PABPN1L, EID2B, BBS4, AGPS, EFCC1, ROBO2, CMTM4, THTPA, ZP4, HIST1H2BE, LOC286238, IFNL2, DGKK, GNGT1, USP17L30, and ERN1. More preferably, the subset of transcriptomics data includes RNA expression levels of at least two genes associated Wnt signaling pathway or pluripotency pathway, which may include SOX2, WNT11, and FZD6. Such obtained subset of transcriptomics data can be further analyzed using the survival prediction model as described above to predict a survival time of the patient.

The inventors further contemplate that, based on the predicted survival time and/or the gene expression data of selected subset of genes, for example, especially SOX2, WNT11, and FZD6, a patient's record can be generated or updated, a new treatment plan can be recommended, or a previously used treatment plan can be updated. For example, where the patient's prognosis is predicted poor (shorter predicted survival time) and the expression level of SOX2 is substantially decreased indicating the de-inhibition of Wnt signaling pathway and metastatic potency of cancer cells, the patient's record can be updated as such and the treatment regimen to the patient can be generated or updated to include a therapeutic agent to inhibit Wnt signaling pathway or increase the SOX2 expression or pre-existing SOX2 activity. Further, the updated or generated treatment regimen may include the treatment timeline that reflect the predicted survival time (e.g., eliminating some choice of treatment plan that may take longer than the expected survival time and modifying the regimen with the treatment that can be finished within 50% of the expected survival time, etc.). In such embodiments, it is also contemplated that the patient's transcriptomics data can be obtained after applying the updated treatment regimen (e.g., at least 5 days after the treatment, at least 10 days after treatment, etc.) to further predict the post-treatment survival time.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Finally, and unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the inventive subject matter and does not pose a limitation on the scope of the inventive subject matter otherwise claimed. No language in the specification should be construed as indicating any non-claimed element essential to the practice of the inventive subject matter.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of generating a survival prediction model for metastatic breast cancer, comprising: obtaining transcriptomics data of a plurality of patients diagnosed with metastatic breast cancer;
clustering the transcriptomics data into a plurality of clusters using complete Pearson correlation;
identifying at least one cluster that is associated with a poor survival of at least some of the plurality of patients by correlating the plurality of clusters with overall survival of the plurality of patients;
generating the survival prediction model predicting a survival time based on expression levels of a plurality of genes in the at least one cluster that is associated with a poor survival of at least some of the plurality of patients; and
wherein the plurality of genes comprise at least one gene associated with WNT signaling pathway or pluripotency pathway.

2. The method of claim 1, wherein the transcriptomics data comprises RNA expression levels of at least 1,000 genes.

3. The method of claim 1, wherein number of the plurality of clusters is determined using elbow method.

4. The method of claim 1, wherein the plurality of clusters is differentially correlated with the overall survival of the plurality of patients.

5. The method of claim 1, wherein the at least one cluster has a hazard ratio is higher than 1.3.

6. The method of claim 1, wherein the plurality of genes are selected among the at least one cluster's transcriptomics data based on a quality of separation of high survivors from low survivors among the plurality of patients in a function of the expression levels of the plurality of genes.

7. The method of claim 1, wherein a number of the plurality of genes is less than 50.

8. The method of claim 1, wherein the plurality of genes are selected from a group consisting of TMEM257, FAM180B, WNT11, CTDSPL, PROK1, GAD2, GRK7, FZD6, KRTAP505, KRT31, PRAMEF12, SYNGR4, SOX2, BHLHA9, POU1F1, KHNYN, CACNA2D4, C3orf36, RHOXF2, PABPN1L, EID2B, BBS4, AGPS, EFCC1, ROBO2, CMTM4, THTPA, ZP4, HIST1H2BE, LOC286238, IFNL2, DGKK, GNGT1, USP17L30, and ERN1.

9. The method of claim 1, wherein the transcriptomics data comprises RNA-seq data.

10. The method of claim 1, further comprising calculating concordance-index of the survival prediction model by comparing the predicted survival time with an actual survival time of the patients.

11. The method of claim 10, wherein the concordance-index is higher than 0.7.

12-19. (canceled)

20. A method of predicting a survival time of a patient diagnosed with metastatic breast cancer, comprising:
obtaining transcriptomic data of a tumor tissue of the patient;
determining RNA expression levels of a plurality of genes from the transcriptomics data;
predicting, using a survival prediction model, the survival time of the patient based on the RNA expression levels; and
wherein at least two genes among the plurality of genes are associated with Wnt signaling pathway or pluripotency pathway.

21. The method of claim 20, wherein transcriptomics data comprises RNA-seq data.

22. The method of claim 20, wherein a number of the plurality of genes is less than 50.

23. The method of claim 20, wherein the plurality of genes are selected from a group consisting of TMEM257, FAM180B, WNT11, CTDSPL, PROK1, GAD2, GRK7, FZD6, KRTAP505, KRT31, PRAMEF12, SYNGR4, SOX2, BHLHA9, POU1F1, KHNYN, CACNA2D4, C3orf36, RHOXF2, PABPN1L, EID2B, BBS4, AGPS, EFCC1, ROBO2, CMTM4, THTPA, ZP4, HIST1H2BE, LOC286238, IFNL2, DGKK, GNGT1, USP17L30, and ERN1.

24. The method of claim 20, wherein the survival prediction model is generated using steps of:
obtaining transcriptomics data of a plurality of patients diagnosed with metastatic breast cancer;
clustering the transcriptomics data into a plurality of clusters using complete Pearson correlation;
identifying at least one cluster that is associated with a poor survival of at least some of the plurality of patients by correlating the plurality of clusters with overall survival of the plurality of patients; and
selecting the plurality of genes from the at least one cluster based on a quality of separation of high survivors from low survivors among the plurality of patients in a function of the expression levels of the plurality of genes.

25. The method of claim 24, wherein the transcriptomics data of the plurality of patients comprises RNA expression levels of at least 1,000 genes.

26. The method of claim 25, wherein number of the plurality of clusters is determined using elbow method.

27. The method of claim 26, wherein the plurality of clusters is differentially correlated with the overall survival of the plurality of patients.

28-43. (canceled)

44. A method of generating or updating a treatment regimen for a patient diagnosed with metastatic breast cancer, comprising:
obtaining transcriptomic data of a tumor tissue of the patient;
determining RNA expression levels of a plurality of genes from the transcriptomics data; predicting, using a survival prediction model, the survival time of the patient based on the RNA expression levels; and
generating or updating the treatment regimen to include at least one agent targeting a pathway element of Wnt signaling pathway or pluripotency pathway.

45-67. (canceled)
